(19) United States
(12) Patent Application Publication
(10) Pub. No.: US 2009/0297610 A1
(43) Pub. Date: Dec. 3, 2009

(54) Composition and system to promote wound healing

(75) Inventors: Diane Ranger, Dana Point, CA (US); Curtis Allen Cluff, Alamitos, CA (US)

Correspondence Address: CRAIN, CATON & JAMES, FIVE HOUSTON CENTER, 1401 MCKINNEY, 17TH FLOOR, HOUSTON, TX, 77010, US

(73) Assignee: COLORESCIENCE, INC., Dana Point, CA (US)

(21) Appl. No.: 12/132,197

(22) Filed: Jun. 3, 2008

(51) Int. Cl. A61K 9/14; A61P 17/02; A61K 33/24; A61K 33/26; A61K 33/30

(52) U.S. Cl. 424/489; 424/642; 424/646; 424/653; 424/617

(57) ABSTRACT

An externally applied non-absorbent composition applied to a wound to promote the healing of skin. The composition contains a plurality of granulated minerals to provide a protective cover and to mask the wound. The composition, together with similar compositions, may be assembled as a system and layered above the wound to better bar undesired particles from contaminating the wound and to camouflage the wound site. The compositions of the system may each be applied via a dispensing brush which retains the composition in a reservoir and dispenses the composition to the skin through the brush bristles, preventing contamination of the composition from the wound.

### CROSS REFERENCE TO RELATED APPLICATIONS

None.

### STATEMENT REGARDING FEDERALLY SPONSORED RESEARCH OR DEVELOPMENT

Not Applicable.

### BACKGROUND OF THE INVENTION

1. Field of the Invention

This invention relates generally to external non-absorbent applications to promote the healing of a wound to the skin.

2. Description of the Related Art

Various compositions and methods are known of applications intended to aid in the body's healing of wounds. These compositions have historically been solely directed to wound fillers which absorb the fluids exuded by the body and which are in turn absorbed into the body These compositions generally are moisturized before application, permitting application as a cream. Often these moisturized compositions include zinc oxide and/or titanium oxide.

Problematically, these applications create additional difficulties. Debris becomes trapped in the moist composition, requiring further cleaning and application. When applied, these compositions also are quite evident on the skin of the patient, drawing often-unwanted attention to the wound. Moreover, the application of these compositions may be via less than sanitary means, such as by hand or by a re-usable brush and powder reservoir, the latter causing contamination of the reservoir contents with each use. Moreover, for various reasons, including the foregoing, the patient may be reticent to reapply the compositions known in the art, which would otherwise aid in wound healing.

Various compositions including minerals are known to be used as color makeup or sunscreens. These products are used to give a desired aesthetic appearance to the skin and/or to camouflage real or perceived skin imperfections.

Problematically these compositions often include chemicals or ingredients that are irritants to compromised skin and therefore generally should be avoided on or around wounded or compromised skin.

It would therefore be an improvement to provide a composition which would promote wound healing, without providing any medicinal aid, which would be applied in dry form to provide a shield to the skin, and which could be applied by a sanitary means.

It would be a further improvement to provide a composition containing powdered minerals which would provide protection to the skin from external factors, such as heat, sun, pollution, dust, dirt and the like, and which could further obfuscate the appearance of the wound.

It would be a further improvement to provide a composition to promote wound healing which would be more readily reapplied.

### SUMMARY OF THE INVENTION

It is therefore a principle object of the present invention to provide a composition that will foster a more sterile environment to promote wound healing after the wound has become minimally exuding, in a more effective and aesthetically desirable manner to traditional wound dressings such as gauze or cotton bandages. The composition is applied externally to form a protective layer over the wounded skin which can create a protective coating environment over the skin for promotion of wound healing, provide an even delivery of minerals over the skin without preventing the absorption of beneficial minerals, prepares minerals to be more water resistant and which provides a more even coat. Moreover, the composition may provide by itself or by a second or third composition camouflaging characteristics, so as to disguise the wound.

The invention includes a composition, a system including two compositions, and a method of use. The composition includes a dry, powdered, non-absorbent substance containing minerals granulated so as to be small enough to spread uniformly over the skin and stay in place but large enough to ensure that the minerals contained therein do not penetrate the skin barrier or are not measurably absorbed into the skin. Such minerals may be sheered or crushed to the appropriate particle size, ideally between 90 nanometers and 50 micrometers in diameter. The substance itself contains a therapeutically-effective amount of mineral composites, which may be zinc oxide, titanium dioxide, iron oxide, bismuth oxychloride, mica, boron nitride, or other similar non-irritating mineral substances that can be crushed or sheered into a fine powder. Depending on the number of layers applied, the substance may be limited, such as to no more than 35% of zinc oxide, titanium dioxide, iron oxide, bismuth oxychloride, aand boron nitride and no more than 70% mica by weight of the composition when applied as a single layer, or when part of a system for applying two composition layers. In all cases, the composition contains no excipient carrier. When used, the composition or compositions may be applied to the skin by a brush containing a reservoir, a dispensing brush, thus precluding the need to insert the contaminated brush ends into a container of composition for further application. The evident camouflaging benefits of the composition are also more likely to encourage regular subsequent applications, thus continuing to promote wound healing.

The foregoing and other objectives, features, and advantages of the invention will be more readily understood upon consideration of the following detailed description of the invention, taken in conjunction with the accompanying drawings.

### BRIEF DESCRIPTION OF THE DRAWINGS

So that the manner in which the described features, advantages and objects of the invention, as well as others which will become apparent, are attained and can be understood in detail, more particular description of the invention briefly summarized above may be had by reference to the embodiments thereof that are illustrated in the drawings, which drawings form a part of this specification. It is to be noted, however, that the appended drawings illustrate only a typical preferred embodiment of the invention and are therefore not to be considered limiting of its scope as the invention may admit to other equally effective embodiments.

FIG. 1 illustrates a minimally-exuding wound on the skin of person.

FIG. 2 illustrates application of a composition of the present invention to the minimally-exuding wound on the skin of person.

FIG. 3 illustrates a dispensing brush for application of a composition of the present invention to the wound on the skin of a person.

### DETAILED DESCRIPTION OF THE PREFERRED EMBODIMENT

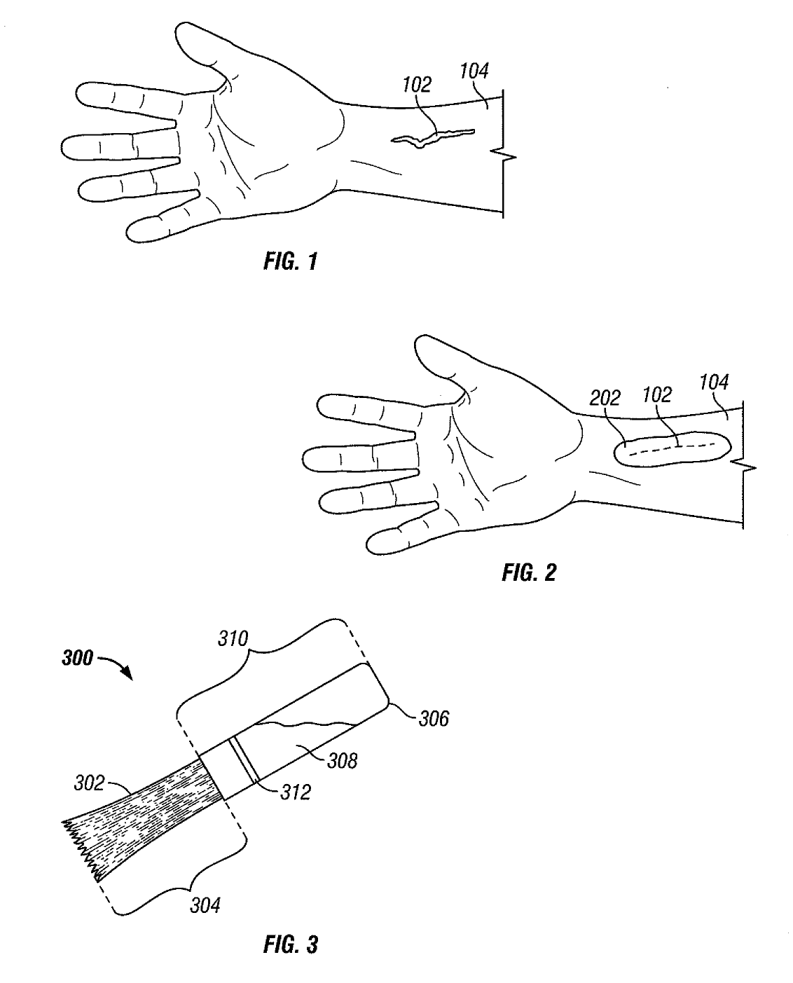

Referring first to FIG. 1, a wound 102 of the type discussed herein is illustrated. No application of the composition of the present invention may be made until the wound is no longer discharging. This may be after the skin 104 has been punctured or cut, whether by outside trauma or by surgery, and has set, that is when the wound is not bleeding or discharging fluids, typically forty-eight (48) to seventy-two (72) hours after treatment. Alternatively, the composition may be for skin that has been compromised, such as with accidental burns or intentional aesthetic chemical peels and/or laser procedures, but not pierced, whether by puncturing or cutting, which application may be nearly immediate.

The composition of the present invention promotes healing of a minimally-exuding wound by providing a protective coating over the wound, rather than intentionally being absorbed into the body. To accomplish this, the composition includes a dry, powdered, non-absorbent substance granulated to 90 nanometers to 50 micrometers in diameter. Granulation is necessary as the substance contains therapeutically-effective amounts of at least two mineral composites. Prior attempts to provide a usable composition did not include the use of a dry, powdered and non-absorbent mineral form. The mineral compositions selected for the present invention are zinc oxide, titanium dioxide, iron oxide, bismuth oxychloride, and mica. If a single composition layer is to be applied, the composition contains no more than 35% by weight of zinc oxide, titanium dioxide, iron oxide, and bismuth oxychloride, and up to 70% by weight of mica, or may be further constrained so that the composition contains no more than 24% by weight of each mineral other than mica such as for a more aesthetically pleasing composition, particularly for variation in color and texture. Notably, the composition contains no excipient carrier. Excipient carriers, which an inactive substance, are often used as a carrier for active ingredients, to promote absorption by the body of the associated composition, to bulk up applications for proper dosing, or to keep an active ingredient in purified, usable form. As a result of the mineral composition and the granulation of the minerals contained therein, the resulting protective layer provides flexible coverage, much like a second skin, over the wound.

Turning to FIG. 2, application on and around the wounded or compromised skin 102 of the composition 202 of the present invention may provide a functional bandage on visible or flexible areas of the skin. The composition provides a physical cover which disguises wounded or compromised skin while simultaneously aiding to keep the wound free of pollutants, elements and other external factors that might otherwise inhibit the skin's natural healing process. Each of the various minerals from which may be included in the composition provides a benefit. Zinc oxide, titanium dioxide, and bismuth oxychloride camouflage the wound site as a functional bandage, thereby creating a protective environment over the skin to aid in the prevention of inhibition of wound healing by outside factors. Additionally, iron oxide and mica provide a protective coating over the wound. Notably, as the composition is intended to cover and conceal the wound, rather than to be absorbed into it, the composition may include color pigmentation to better mask the wound and to blend to the adjacent skin color. Beneficially, this camouflaging is accomplished without using absorbing, liquid-based cosmetics that could inhibit or harm skin function.

This composition may contain other constituents in minimal quantities, generally less than three percent (3%) by weight for any constituent to ensure the coating/wound dressing function is not compromised. The composition may include a combination of cyclopentasiloxonane, dimethicone crosspolymer and/or dimethiconol, which would provide for an increase in water resistance and for even delivery of minerals over the skin while allowing other beneficial ingredients to penetrate. Melissa officinailis leaf aloe vera oil, tea tree oil, lavender oil, tangerine oils, canana odorata (ylang ylang), santalum album (sandlewood), and/or rose geranium phospholipids may be included to provide soothing against irritation and other benefits. Similar, vitamin E may be included to provide skin health benefits. Oat beta glucan may be included to provide antioxidant qualities and to reduce skin redness. Blue algae extract (halsea ostrearia) may be incorporated to moderate oiliness of skin and to tighten its feel. Compounds may be used to dissolve non-mineral ingredients. These compounds may include citrus grandis (grapefruit) extract green tea extract propylene glycol), butylene glycol, and phenoxyethanol. Stability or homogenization may be accomplished by including constituents such as beta carotene (carrot seed oil), stearic oxide, dimethicone, cylcopentasiloxane, vinyldimethicone, and calcium silicate.

Unlike the prior art which provides for penetration of skin and stimulation of the healing process, for promotion of collagen and elastin, and for improved wound reepithelialization, the use of crushed minerals in a dry crushed powder form with no excipient carriers does not penetrate the skin. The minerals in the composition are not intended to stimulate skin or cell function, but to provide a clean environment and to help keep the wound free from pollutants that it might otherwise contact.

In an alternative embodiment, a plurality of layers of composition utilized to provide a system to promote wound healing. The system thus includes a first composition and at least a second composition and potentially a third composition. The first composition, second composition, and, if applicable, the third composition, each contain a dry, powdered, non-absorbent substance granulated to 90 nanometers to 50 micrometers in diameter containing no excipient carrier. The compositions differ in the mineral substances used therein. The first substance contains therapeutically-effective amounts of zinc oxide, titanium dioxide, iron oxide, bismuth oxychloride, all in amounts not more than 35% by weight of the first composition and mica, in an amount of not more than 70% by weight of the first composition. This may optimally include amounts not more than 18% by weight of the first composition, particularly where coloring and texture variation are desired. The second substance contains therapeutically-effective amounts of zinc oxide, titanium dioxide, and bismuth oxychloride, all in amounts not more than 35% by weight of the second composition and mica in an amount not more than 70% by weight of the second composition. The third substance, if used, contains therapeutically-effective amounts of zinc oxide and titanium dioxide, all in amounts not more than 35% by weight of the third composition. Ideally, the outermost composition has coloring and texture consistent with the skin adjacent the wound so to as to obfuscate the wound. In the case of both the second and third substances, this may optimally include zinc oxide, titanium dioxide, and bismuth oxychloride in amounts of not more than 24% by weight and mica in an amount of not more than 70% by weight of the second or third composition, particularly where coloring and texture variation are desired.

Additionally, applicators for the various compositions may be included in a system to provide ease of application. Such applicators may include the dispensing brush depicted in FIG. 3. As depicted, the dispensing brush 300 may have bristles 302 at its first end 304 and a composition reservoir 306 containing a particular composition 308 at its second end 310. The dispensing brush composition reservoir 306 is adapted to communicate the particular composition 308 to the dispensing brush bristles 302. This may be accomplished by a filter 312. Bristles 302 may be detachable from brush 300 for replacement with new bristles 302, thus permitting re-use of the brush. Thus, the system may include as many dispensing brushes as compositions used.

In operation, the first composition would be applied, preferably by the dispensing brush, after the wound was sufficiently dry, or at least discharging only minimally. Once a layer was formed, the second composition would be applied. If applicable, the third layer would be applied after the formation of the layer using the second composition.

The foregoing disclosure and description of the invention is illustrative and explanatory thereof. Various changes in the details of the illustrated process may be made within the scope of the appended claims without departing from the spirit of the invention. For example, silicon powder may be used to promote agglomeration of one or more of the compositions. The present invention should only be limited by the following claims and their legal equivalents.

I claim:

1. A composition to promote healing of a minimally-exuding wound on the skin of a person, comprising:
a) a first dry, powdered, non-absorbent substance granulated to 90 nanometers to 50 micrometers in diameter,
b) said first substance containing therapeutically-effective amounts of at least two mineral composites selected from the group of zinc oxide, titanium dioxide, iron oxide, bismuth oxychloride, and mica,
c) said composition containing no more than 35% by weight of any of said zinc oxide, titanium dioxide, iron oxide, bismuth oxychloride and no more than 70% by weight of any of said mica,
d) said composition containing no excipient carrier.

2. A system for promoting healing of a minimally-exuding wound on the skin of a person, comprising:
a first composition,
said first composition containing a first dry, powdered, non-absorbent substance granulated to 90 nanometers to 50 micrometers in diameter,
said first substance containing therapeutically-effective amounts of zinc oxide, titanium dioxide, iron oxide, bismuth oxychloride, and mica,
said first composition containing no more than 35% of said zinc oxide by weight,
said first composition containing no more than 35% of said titanium dioxide by weight,
said first composition containing no more than 35% of said iron oxide by weight,
said first composition containing no more than 35% of said bismuth oxychloride by weight,
said first composition containing no more than 70% of said mica by weight, and
said first composition containing no excipient carrier.
a second composition,
said second composition containing a second dry, powdered, non-absorbent substance granulated to 90 nanometers to 50 micrometers in diameter,
said second substance containing therapeutically-effective amounts of zinc oxide, titanium dioxide, bismuth oxychloride, and mica,
said second composition containing no more than 35% of said zinc oxide by weight,
said second composition containing no more than 35% of said titanium dioxide by weight,
said second composition containing no more than 35% of said iron oxide by weight,
said second composition containing no more than 35% of said bismuth oxychloride by weight,
said second composition containing no more than 70% of said mica by weight, and
said second composition containing no excipient carrier.

3. The system of claim 2, wherein:
the outermost composition having color and texture consistent with said skin, said second composition obfuscating said wound.

4. The system of claim 3, further comprising:
a third composition,
said third composition containing a third dry, powdered, non-absorbent substance granulated to 90 nanometers to 50 micrometers in diameter,
said third substance containing therapeutically-effective amounts of zinc oxide and titanium dioxide,
said third composition containing no more than 35% of said zinc oxide by weight,
said third composition containing no more than 35% of said titanium dioxide by weight, and
said third composition containing no excipient carrier.

5. The system of claim 4, further comprising:
a first dispensing brush, said first dispensing brush having bristles at its first end and a composition reservoir containing said first composition at its second end, said first dispensing brush composition reservoir adapted to communicate said first composition to said first dispensing brush bristles;
a second dispensing brush, said second dispensing brush having bristles at its first end and a composition reservoir containing said second composition at its second end, said second dispensing brush composition reservoir adapted to communicate said second composition to said second dispensing brush bristles; and
a third dispensing brush, said third dispensing brush having bristles at its first end and a composition reservoir containing said third composition at its second end, said third dispensing brush composition reservoir adapted to communicate said third composition to said third dispensing brush bristles.

6. The system of claim 4, further comprising:
a dispensing brush, said dispensing brush having bristles at its first end and a composition reservoir adapted to contain one of said first, second or third compositions at its second end, said dispensing brush composition reservoir adapted to communicate said first, second or third composition to said dispensing brush bristles.

7. A method to promote healing of a minimally-exuding wound on the skin of a person, comprising:
applying a first composition containing a first dry, powdered, non-absorbent substance about said wound, said first substance granulated to 90 nanometers to 50 micrometers in diameter, said first substance containing therapeutically-effective amounts of at least two mineral composites selected from the group of zinc oxide, titanium dioxide, iron oxide, bismuth oxychloride, and mica, said first composition containing no more than 35% by weight of any of said zinc oxide, titanium dioxide, iron oxide, bismuth oxychloride and no more than 70% by weight of any of said mica, said first composition containing no excipient carrier.

8. The method of claim 6, further comprising;
applying a second composition containing a second dry, powdered, non-absorbent substance about said wound, said substance granulated to 90 nanometers to 50 micrometers in diameter, said substance containing therapeutically-effective amounts of at least two mineral composites selected from the group of zinc oxide, titanium dioxide, iron oxide, bismuth oxychloride, and mica, said composition containing no more than 35% by weight of any of said zinc oxide, titanium dioxide, iron oxide, bismuth oxychloride and no more than 70% by weight of any of said mica, said second composition containing no excipient carrier.

9. The method of claim 8, farther comprising:
applying a third composition containing a third dry, powdered, non-absorbent substance about said wound, said third substance granulated to 90 nanometers to 50 micrometers in diameter, said third substance containing therapeutically-effective amounts of at least two mineral composites selected from the group of zinc oxide, titanium dioxide, iron oxide, bismuth oxychloride, and mica, said third composition containing no more than 35% by weight of any of said zinc oxide, titanium dioxide, iron oxide, bismuth oxychloride and no more than 70% by weight of any of said mica, said third composition containing no excipient carrier.
